(19) United States
(12) Patent Application Publication
(10) Pub. No.: US 2020/0344960 A1
(43) Pub. Date: Nov. 5, 2020

(54) NUTRIENT MEDIUM FOR AUTOMATED CULTIVATION OF PLANTS

(71) Applicant: RoBoTEC PTC GmbH, Bremen (DE)

(72) Inventor: Friederike Von Rundstedt, Bremen (DE)

(73) Assignee: RoBoTEC PTC GmbH, Bremen (DE)

(21) Appl. No.: 16/959,719

(22) PCT Filed: Jan. 10, 2019
(86) PCT No.: PCT/EP2019/050511

§ 371 Date: Jul. 2, 2020

(30) Foreign Application Priority Data
Jan. 10, 2018 (DE) 10 2018 100 485.0

(51) Int. Cl. A01G 24/40; A01G 24/22; A01G 24/12; A01G 24/30; A01G 24/25; A01G 31/02

(52) U.S. Cl. CPC A01G 24/40; A01G 24/22; A01G 31/02; A01G 24/30; A01G 24/25; A01G 24/12

(57) ABSTRACT

A nutrient medium for the cultivation of plants, containing (a) 3 g/l to 18 g/l agar and (b) 0.5 g/l to 3 g/l carrageenan, as well as plant plugs which contain such a nutrient medium, a method for the production of the plant plugs, and a method for the automated or semi-automated cultivation of plants using the plant plugs.

### CROSS REFERENCE TO RELATED APPLICATIONS

This patent application is the US National Phase of and claims priority on and the benefit of International Application No. PCT/EP2019/050511 having an international filing date of 10 Jan. 2019, which claims priority on German Patent Application No. 10 2018 100 485.0 having a filing date of 10 Jan. 2018.

### BACKGROUND OF THE INVENTION

### Technical Field

The invention relates to a growth medium for plants and plant plugs produced therefrom (“in vitro plugs”).

### Prior Art

In the area of vegetative plant cultivation, especially the propagation of fruit-bearing and esthetic plants, it is becoming increasingly important to ensure a standardized and pathogen-free production of new plants. Especially the use of soil substrates is increasingly associated with problems owing to a growing internationalization of the trade. For instance, various countries prohibit the import of plants which were cultivated on soil substrates, since it is hardly possible to ensure their pathogen-free status. The use of soil substrates is therefore frequently associated with an increased complexity when, for example, it is necessary to establish various growth substrates for different countries or to obtain special permits for their import.

For these reasons, plant breeders are increasingly relying on in vitro substrates for plant propagation, based on gellan gum for example. The in vitro substrates generally contain standardized amounts of nutrients and can be sterilized, meaning that it is possible to guarantee their pathogen-free status. In the case of regenerative propagation, individual shoots are usually applied to the in vitro substrate by hand. Once a fine root system has formed, it is then possible—again by hand—to further process the plant unit formed from shoot and substrate, for example by introducing both into a soil substrate. However, this approach is labor-intensive, time-consuming and cost-intensive. Furthermore, manual processing is only standardizable to a limited extent.

PCT/EP2017/000922 describes a device which is capable of carrying out the above-described propagation steps in an automated manner. First of all, the device grips an individual plant to be propagated by a first gripper. Thereafter, the individual plants hanging on the first gripper are cut specifically into multiple clones and the individual clones are taken away in an automated manner by a second gripper for further processing.

To be able to likewise automate the subsequent steps—especially the application of the clones to a plant plug composed of a growth medium and the further handling of said plant plug—there is a need for a growth medium which, firstly, is sufficiently solid to allow handling by grippers of a device and, secondly, does not prevent the formation of an adequate root system. Currently known in vitro growth media do not meet these requirements because they do not have sufficient stability.

### BRIEF SUMMARY OF THE INVENTION

The object is achieved by a growth medium for the cultivation of plants, characterized in that the growth medium comprises (a) 3 g/l to 18 g/l agar and (b) 0.5 g/l to 3 g/l carrageenan, and by plant plugs comprising said growth medium.

In particular, the invention provides in a first aspect a growth medium for the cultivation of plants, characterized in that it comprises (a) 3 g/l to 18 g/l agar; and (b) 0.5 g/l to 3 g/l carrageenan. In the context of the invention, it was found that, surprisingly, this mixture of the gelling agents agar and carrageenan has a sufficient strength for handling by machine grippers and, at the same time, allows the formation of the necessary root system, even though the individual gelling agents alone in comparable concentrations would not generate a sufficient gelling effect. The growth medium according to the invention is thus suitable for both the manual and the automatic cultivation and propagation of plants. In the growth medium according to the invention, it is possible to carry out all important cultivation steps, including the development, the propagation, the protection and the optimization of the plants.

Compared to the growth media and methods for plant cultivation that are currently used, the invention is especially distinguished by more rapid growth of the plants, since their growth is not inhibited in a plant plug produced using the growth medium according to the invention. The plants cultivated in this manner develop in a uniform manner and develop into robust plants. Furthermore, the cultivation methods automatable according to the invention are associated with a good planability of culture and sale parameters, cost and time savings due to automation, reduced manual work, assured plant health and higher growth rates. The growth media and plant plugs according to the invention are globally exportable, since they are sterile and disease-free and plant health is hence certifiable. By means of the plant plugs and a corresponding plant tray, young plants can also be protected from drying out and damage. Lastly, the plants in the plant plugs can also be more easily adapted to nonsterile greenhouse conditions after transplantation, meaning that waste is reduced.

The growth medium according to the invention is especially a medium suitable for the vegetative propagation of plants. At the same time, the medium can, however, also be used for the generative propagation, especially the seeding, of plants. In contrast to generative propagation, vegetative propagation is asexual and dependent on plant cultivation from plant parts, for example cuttings. It is known to experts that growth media contain—besides the constituents described here as essential—further additives, such as nutrients and the like. These are adjustable to the plant species in question, to the purpose of cultivation and to the type of cultivation (vegetative/generative) and are therefore variable. Suitable additives and combinations of additives are described herein at another point.

The plants cultivable and propagable according to the invention can be any plants which can grow on a substrate comprising the gelling agents agar and carrageenan. In particularly preferred embodiments, the products and methods according to the invention are suitable for the cultivation of ornamental plants, herbaceous perennials and/or woody plants. Preferred ornamental plants encompass, for example, Phalaenopsis (orchids), Anthurium and Spathiphyllum. Preferred herbaceous perennials encompass, for example, Echinacea, Helleborus and Heuchera. Preferred woody plants encompass, for example, Lycium, Paulownia and Vaccinium. It is evident that the use of the products and methods according to the invention is not limited to these plants.

The term “cultivation” refers to the establishment and maintenance of conditions which ensure, i.e., promote and/or allow, plant growth. This encompasses conditions which ensure propagation. Preferably, the methods and products according to the invention are used for the propagation of plants, i.e., the cultivation of a plant shoot from a seed or the cultivation of a plant shoot from a clone.

As mentioned, the growth medium comprises 3 g/l to 18 g/l agar. Preferably, the growth medium comprises from 5 g/l to 12 g/l agar, more preferably from 5.8 g/l to 9.5 g/l. In one embodiment, the growth medium comprises agar in a concentration of from 5.8 g/l to 7 g/l. For example, agar can be present in the growth medium in a concentration of about 6.4 g/l.

Agar (or: agar-agar) is a structural carbohydrate in the cell walls of certain algae that comprises galactose polymers and can form gels. Sources for obtaining agar and methods for producing agar are known to experts. For example, agar can be obtained from the cell walls of some algae species, for example from red algae. In agar, the strong gel former agarose is responsible for the gelling ability. In the EU, agar is authorized as a food additive under number E 406. Whereas agar already gels at 45° C., it is highly temperature-resistant, meaning that it can be sterilized even at high temperatures. Furthermore, it is also repeatedly successively sterilizable without substantial loss of strength. Agar is commercially available in sufficient quantities (e.g., as CERO Agar Agar Powder, type 8925 X; CERO Agar Agar Gracellaria Powder, type 8925 Q; 1-Plant Agar 1200 g/cm²; I.A.-Mikro Agar 880 g/cm²; Vitro Agar 1200-900 g/cm²; Vitro A1 Agar 1200 g/cm²; Gelrite; Gellan Gum type 2). Preferably, the agar has a gel strength (determined according to the Nikkan-Kobe test; 1.5%, 15 h) of from 800 to 1300 g/cm², preferably from 800 to 1000 g/cm², more preferably 850 to 960 g/cm², yet more preferably 890 to 930 g/cm², for example 910 g/cm². Methods for determining gel strength are known to experts and encompass the Nikkan-Kobe test.

Furthermore, the growth medium comprises 0.5 g/l to 3 g/l carrageenan. Preferably, the growth medium comprises 1 g/l to 2.2 g/l carrageenan, more preferably 1.3 g/l to 1.9 g/l carrageenan. For example, carrageenan can be present in the growth medium in a concentration of about 1.6 g/l.

Carrageenan is likewise a gelling agent composed of long-chain carbohydrates which occur in red algae cells (Irish Moss Chondrus crispus) (synonym: Danish agar). In the EU, carrageenan is authorized as a food additive under number E 407. Carrageenan is commercially available in sufficient quantities (e.g., as CEROGEL Carrageenan, type 8886). Preferably, the carrageenan has a gel strength (determined according to the Nikkan-Kobe test; 1.5%, 20° C.) of from 400 to 1000 g/cm², more preferably 500 to 900 g/cm², yet more preferably 600 to 800 g/cm². The carrageenan can be immediately used in the growth medium according to the invention after its isolation from red algae extracts. Since, within the carrageenan extract, the kappa fraction in particular is distinguished by a good gelling ability, the carrageenan preferably comprises a high proportion of kappa-carrageenan (κ-carrageenan). For example, the carrageenan can comprise 80% kappa-carrageenan or more, preferably 90% kappa-carrageenan or more. Preferably, the carrageenan used in the growth medium according to the invention is kappa-carrageenan.

In a preferred embodiment, the medium comprises from 3 to 10 g/l agar and 0.5 to 3 g/l carrageenan, more preferably from 5 g/l to 8 g/l agar and 1 g/l to 2.2 g/l carrageenan. For example, the medium can comprise about 6.4 g/l agar and 1.6 g/l carrageenan. Surprisingly, the two components agar and carrageenan in these concentrations already synergistically generate a gel strength suitable for automatic handling.

The medium can comprise agar and carrageenan in a ratio in the range from 6:1 to 2:1 for example, preferably in the range from 5:1 to 3:1, more preferably in the range from 4:1 to 3.5:1.

In a preferred embodiment, the medium is a sterile medium. Methods for sterilization are known to experts. The growth medium can, for example, be sterilized by steam pressure sterilization (autoclaving), microwave sterilization, irradiation or sterile-filtration. It is evident that the last-mentioned method is only suitable for sterilizing the medium in a form that is still liquid. Preferably, sterilization and gelling/solidification of the growth medium are interconnected. Therefore, steam pressure sterilization and microwave sterilization are particularly preferred.

The growth medium is preferably free of constituents which could lead to microbial contamination of the plants. The growth medium is therefore preferably free of soils (soil-free). The term “soils” encompasses, for example, humus, potting soil, peat and the like.

It is known to experts that the gelling agents agar and carrageenan only gel from certain temperatures, thereby giving the growth medium its more solid form needed for (automated) use as plant plugs. The growth medium according to the invention can therefore have a liquid form or a more solid or solid form. Suitable gel strengths which are to be regarded as “solid” herein are defined herein at another point. Whereas the growth medium in solid form is preferred according to the invention, the invention also encompasses liquid growth media. For example, it is conceivable that the growth medium is transported in liquid form. Liquid growth medium can also be used for producing the plant plugs, for example by pouring into appropriate molds.

Agar and carrageenan gel at temperatures from 35-41° C. (agar). However, this does not rule out the possible use of higher temperatures for the gelling of the growth medium, for example for simultaneous sterilization. Experts are capable of ascertaining suitable gelling temperatures. In contrast to other gelling agents, the agar-and-carrageenan mixture according to the invention is repeatedly heatable without substantial loss of quality and hence, for example, also repeatedly sterilizable if necessary.

The growth medium according to the invention is distinguished by having a gel strength which makes it possible to grip and move by machine plant plugs generated using the growth medium and which, at the same time, does not prevent the plants from forming a sufficiently dense root system. The growth medium has especially a gel strength of from 400 to 1200 g/cm², preferably 600 to 1000 g/cm².

In a particularly preferred embodiment, the growth medium comprises a shape-stabilizing component. In addition to the agar and the carrageenan, said shape-stabilizing component contributes to preserving the shape of the plant plug even under mechanical stress, such as the automated movement of the plug, and over a relatively long cultivation period. This additional component is not absolutely necessary for achieving the advantages according to the invention, but can contribute to optimizing the plant plug and growth medium according to the invention. The shape-stabilizing component can be a water-soluble and/or a water-insoluble component.

In preferred embodiments, the shape-stabilizing component is a water-insoluble component. Said component stabilizes a plant plug produced from the growth medium according to the invention and ensures that the root system formed within a plant plug does not collapse when the plug is watered and the gelling agents are thereby dissolved over time. This regularly occurs in the course of the cultivation time when a plant plug is, under greenhouse conditions, introduced into a depression in the next largest soil substrate and watered there. The water-insoluble component is therefore a component not dissolvable by water, especially a network not dissolvable by water.

The water-insoluble component is present in the plant plug (and optionally also already in the liquid growth medium) in the form of a network. This equally applies to the water-soluble component described in more detail below. The network forms a scaffold to support a root system which has formed or is to be formed. The network can arise within the growth medium or plant plug from the water-insoluble component. Alternatively, the network can be added already in the form of a network to the growth medium or plant plug. The network will generally occupy an outer volume which substantially corresponds to that of a plant plug, for example is at most 40%, preferably at most 30%, more preferably at most 20% smaller. At the same time, the network has pores and crosslinked cavities. Within the pores and crosslinked cavities, it is possible for a root system to form.

The water-insoluble component is preferably selected from the group consisting of plant parts, plastics, minerals, mixtures thereof and materials produced therefrom.

Suitable plant parts are, for example, parts of jute, luffa, flax, banana, coconut, Sphagnum moss, argan fruit shells, bark, cork, hemp, and further plant parts or plants, especially fibrous or fiber-containing plant parts or plants, particular preference being given to fibrous or fiber-containing parts of jute, hemp, flax and coconut, i.e., jute, hemp, flax and coconut fibers.

Suitable plastics encompass, for example, polyurethane (e.g., in the form of polyurethane foam) and bioplastics, such as polylactides (PLA; polylactic acids).

Polyurethane, preferably polyurethane foam, is generally produced from a polyol component and an isocyanate component, which are mixed and foamed cold within a mold. Thereafter, the solidified polyurethane (foam) can be cut into smaller parts in the shape and size of a plug. In a mold which is approximately complementary in shape and size, the liquid growth medium according to the invention can be poured over said parts. Suitable polyurethane foams are known to experts and commercially available (e.g., Oasis, Smithers-Oasis Germany GmbH, D-67269 GrUnstadt, and BVB Sublime, BVB Substrates NL-2678 PS De Lier). It has been found that polyurethane foams having a pore size of from 2.00 to 0.05 cm allow particularly good rooting and simultaneous stabilization, and so such polyurethane foams are particularly preferred according to the invention. This equally applies to other shape-stabilizing components used in the context of the invention. The polyurethane foam is preferably a soft foam. Soft foams have the advantage that they remain flexible (soft) after drying and do not become rigid. This, too, equally applies to other shape-stabilizing components, i.e., the shape-stabilizing component is preferably a foam having pores, preferably having a pore size of from 2.00 to 0.05 cm.

Polylactides (PLAs) suitable for the invention have comparable properties to the above-described polyurethanes. Such PLAs are known to experts. Furthermore, the PLAs have a high level of biocompatibility and can be biodegradable. The polylactide is therefore preferably a biodegradable PLA. Furthermore, the polylactide can be a polylactide which is printable by means of 3D printing. In this way, it is, for example, possible to print a shape-stabilizing polylactide scaffold by means of 3D printing and to subsequently pour the growth medium according to the invention over it.

Suitable minerals encompass, inter alia, vermiculite, other silicon compounds, clay minerals and perlites. Materials which are produced from the aforementioned substance groups and which can be used for the growth medium according to the invention are, for example, rock wool, pulp and the like.

From plants, preference is given to using dried plant parts, for example dried parts of fruit (luffa) or the like. Preference is given to the water-insoluble component luffa. These constituents are rich in cellulose. It is evident that dried plant parts can be comminuted in a suitable manner, for example by pestling, before use thereof. As described above, the water-insoluble component can, however, also be directly used in an uncomminuted form, in the form of a network. For example, a portion can be separated from a dried luffa fruit, which portion has approximately the outer volume of a plant plug. In a mold approximately complementary in shape and size, the liquid growth medium can subsequently be poured over the portion. In this process, the liquid growth medium runs into the pores and crosslinked cavities in the network and can subsequently be solidified there by heating. Alternatively, plant fibers, such as jute, hemp, flax or coconut fibers, can, for example, be brought into a plug shape by entangling the fibers and then, as described above, the liquid growth medium can be poured over them in a mold.

What is common to all the water-insoluble components which are mentioned above and suitable according to the invention is that they are capable of maintaining the structure of a plant plug even after the dissolution of the gelling agents agar and carrageenan. The water-insoluble components thus serve to stabilize the root system formed. What are particularly suitable for this purpose are components, for example compounds or mixtures of compounds, which are fiber-containing or form fibers. The components, especially fiber-containing components, will generally form a network within a plant plug or be already present in the form of an (endogenous) network. In other words, the water-insoluble component is a network or a network former. As a result of the stabilizing network, the water-insoluble component is capable of preventing a complete collapse of the plant plug and the root system after the dissolution and removal (e.g., draining) of the water-soluble gelling agents agar and carrageenan.

Preferably, the outer volume of the plant plug is reduced by less than 60%, preferably less than 40%, more preferably by less than 20%, in the case of a dissolution and removal of the gelling agents agar and carrageenan owing to a stabilizing action of the water-insoluble component. This means that a plant plug volume of, for example, 100 cm³ (100%) is stabilized by the water-insoluble component such that said volume is, as a result of the addition of water and/or the dissolution and removal of the gelling agents agar and carrageenan, reduced at most to a volume of 40 cm³ (40% of the original volume), preferably 60 cm³ (60% of the original volume), more preferably 80 cm³ (80% of the original volume).

In connection with the invention, “water-insoluble” means that water has no influence or only a slight influence on the integrity of the component and of structures in the plant plug that are formed therefrom. Self-evidently, this does not rule out that the component and structures formed therefrom can change their shape as a result of the loss of other water-soluble components (such as agar and carrageenan) in the plant plug. For example, as a result of the loss of agar and carrageenan, a network which has formed can slump slightly (i.e., within the aforementioned volumetric limits) owing to the influence of gravity. Furthermore, the term also does not rule out that the component can swell as a result of contact with water, so long as the network structure is substantially maintained (in a stabilizing manner and within the specified volumetric limits).

It is evident that the network may be more greatly compressed by other mechanical or biological influences. Since—precisely in nonsterile greenhouse conditions—it can be assumed that the network is more greatly affected by such influences over time and is also no longer required for stabilization once the root system has sufficiently connected to the nonsterile soil substrate surrounding the plant plug, the above statements relating to stabilization by the network only concern the first six weeks after the transfer of a plant plug into a soil substrate.

As an alternative or in addition to the above-described water-insoluble, shape-stabilizing component, the growth medium can also comprise a shape-stabilizing water-soluble component. Suitable water-soluble components encompass, for example, hydroxyalkylcellulose and gelatin, especially gelatin containing colloidal silver. The hydroxyalkylcellulose is preferably selected from the group consisting of hydroxymethylcellulose and hydroxyethylcellulose, particular preference being given to the former.

Suitable methods for producing hydroxyalkylcelluloses are known to experts. For example, hydroxyethylcellulose (HEC) is a reaction product of ethylene oxide with cellulose which is alkalized and matured by controlled oxidative chain degradation. Here, it is not only the three free OH groups of the glucose unit, preferably the primary hydroxyl group on the C-6 atom, which can react, but also the terminal OH group on the glycolic substituent. Therefore, the so-called degree of molar substitution (MS), the number of ethylene oxide molecules reacted per glucose unit, is two to five times the so-called average degree of substitution (DS). The DS is understood to mean the number of derivatized hydroxyl groups per glucose unit of the cellulose. Level and uniformity of substituent distribution on the polymer chain determine solvation and hence the possibility of producing solutions and hydrogels which are virtually free of suspended particles. The HEC types with differing thickening action are generated in the production process in the maturation of the cellulose. Suitable HECs are known to experts and commercially available and encompass, for example, PHRIKOLAT HEC 100, a highly viscous hydroxyethylcellulose with 4500-6500 mPas in 1% solution (Brookfield). Further suitable hydroxyalkylcelluloses encompass, for example, methylcellulose, Sigma, prod. Nos. M0262, M0387, M0512, M6385 and M7140, CAS NUMBER: 9004-67-5; synonym: methylcellulose A, methylcellulose ether. The hydroxyalkylcelluloses preferably have a viscosity of 400 to 10 000 mPa·s in 2% aqueous solution. According to the invention, the cellulose has alkoxy substitutions, preferably methoxy substitutions, of from 27.5% to 31.5% by weight.

The hydroxyalkylcellulose can be added to the liquid growth medium according to the invention and be solidified together with the growth medium in the desired shape and size. Preferably, the growth medium comprises hydroxyalkylcellulose in a concentration of from 5% to 15%.

What can likewise be used for stabilization is gelatin, preferably a gelatin foam. Here, what is especially preferred is to use gelatin portions which have a smaller size than a plant plug defined herein and to pour the growth medium according to the invention over said portions. Methods for producing suitable gelatin portions, for example gelatin cubes, are known to experts. Furthermore, gelatin portions are also commercially available. It is envisaged to preferably use gelatin portions which contain colloidal silver and thereby display an additional sterilizing action. Appropriate gelatin cubes are, for example, available as “Gelatamp” (Roeko; Coltene, gelatin sponge containing 5% colloidal silver, y-sterile) and “Gelita-Spon®” (Gelita medical; for example cube, 10×10×10, 50, GS-310, Art. 00715118, without silver additive, foamed gel).

As mentioned above, the growth medium comprises further additives, such as, for example, nutrients. The growth medium can, for example, contain nutrients such as, for example, macronutrients and micronutrients; vitamins, phytohormones, further gelling agents, sugar and/or others. All the additives promote or support the growth of the various plant species. This can occur in different ways. For instance, the additives can directly support plant growth, for example by providing building blocks for the formation of cells and the like, or they can only indirectly support plant growth, for example by preventing or curbing the growth of competing organisms, such as bacteria. The additives are each selected and combined depending on the plant species to be cultivated.

Whereas it is possible, as described above, to add additives which prevent or curb the growth of competing organisms, such as bacteria, fungi and the like, to the growth medium, it was found that, surprisingly, the mixture of the gelling agents agar and carrageenan that is defined herein likewise inhibits bacterial growth. The addition of relevant additives is therefore not absolutely necessary according to the invention.

Suitable nutrients encompass macronutrients, such as nitrogen (N), phosphorus (P), potassium (K), calcium (Ca), magnesium (Mg) and sulfur (S), the macronutrients preferably being present in the growth medium in the form of chemical compounds which contain the respective macronutrient and make it available for the plants. The growth medium comprises, for example, macronutrients in the form of KNO₃, NH₄NO₃, MgSO₄×7 H₂O, KH₂PO₄ and/or CaCl₂)×2 H₂O. The concentration is from 100 to 2000 ppm.

Furthermore, suitable nutrients encompass micronutrients, such as boron (B), iron (Fe), iodine (I), cobalt (Co), copper (Cu), manganese (Mn), molybdenum (Mo), sodium (Na) and zinc (Zn), the micronutrients preferably being present in the growth medium in the form of chemical compounds which contain the respective macronutrient and make it available for the plants. The growth medium comprises, for example, macronutrients in the form of MnSO₄×H₂O, ZnSO₄×7 H₂O, H₃BO₃, Na₂MoO₄×2 H₂O, CuSO₄×5 H₂O, KJ and CoCl₂×6 H₂O and NaFe EDTA. The concentration in the growth medium is from 0.01 to 50 ppm.

Suitable vitamins and vitamin-like substances which are comprised by the growth medium are, for example, thiamine, nicotinic acid, pyridoxine, glycine and myo-inositol. The concentration in the growth medium is from 0.01 to 200 ppm.

Phytohormones control certain growth processes in plants, and so their admixture and concentration is selected depending on the plant species and the purpose of growth (root formation, shoot formation, branching, extension, etc.). Experts are capable of making an appropriate selection of phytohormones, and of the other additives defined herein, and of choosing suitable concentrations in each case. The growth medium can, for example, comprise phytohormones from the following groups of active ingredients: abscisic acid, auxins, cytokinins, gibberellins. The phytohormone(s) is/are present in the growth medium as, for example, indole-3-acetic acid (IAA), 4-(indo-3-yl)butyric acid (IBA), 1-naphthylacetic acid (NAA), 6-benzylaminopurine (BAP), kinetin (KIN), zeatin (ZEA) or 2-isopentenyladenine (2iP). The concentrations in the growth medium are from 0.001 to 50 ppm.

Furthermore, the growth medium can comprise sugar. The sugar is preferably added in the form of sucrose, glucose or/and fructose. The concentrations in the growth medium are 1 to 50 g/L growth medium.

Self-evidently, what can be provided is that the growth medium has a certain pH, for example a pH at which the plant in question optimally thrives.

Experts can select further additives and add them to the growth medium depending on the plant species and intended use.

In a related aspect, the invention provides plant plugs for the cultivation of plants, characterized in that the plant plug comprises a growth medium according to the invention.

Plant plugs (commonly referred to as in vitro plugs) are generally relatively small shaped bodies composed of a growth medium that serve for the cultivation and propagation of plants in a very early developmental stage. Plant plugs generally have a consistency which allows a manual or machine transfer of the plugs into other cultivation vessels or transport or processing units. Owing to their small size and transferability, plant plugs can be suitable for medium- and high-throughput methods in plant cultivation and can also be used for the space-saving transport of plants in an early developmental stage.

The plant plugs according to the invention are suitable both for the manual cultivation of plants and for automated or semiautomated and machine cultivation of plants. Preferably, the plant plugs are for the automated or semiautomated cultivation of plants. In this connection and within the meaning of DIN V 19233, “automated” means that the cultivation is carried out by an apparatus which is equipped such that the apparatus works as intended (i.e., achieves a step forward in the cultivation of plants) without any participation at all by a person or with some participation by a person. In other words, the apparatus works autonomously. In the case of the automated or semiautomated cultivation of plants, a plant shoot or plant clone in particular is applied to a plant plug in an automated manner; the plant unit formed from plant part and plant plug is transferred into another device, another device part and/or a container; and/or the plant unit is transferred into a larger plant plug or a soil substrate, it being possible for the larger plant plug to be a plant plug according to the invention or a different type of plant plug.

The automated “application” of a plant shoot or plant clone to a plant plug encompasses various application techniques, for example application by laying or application by pressing. It is likewise conceivable to apply a plant shoot or plant clone to a plant plug by insertion into a depression (e.g., a slit) introduced into the plant plug beforehand. At the same time, the plant shoot or plant clone is generally oriented such that its leaves point away from the plant plug and its stalk is in direct contact with the plant plug.

The plant plug is preferably producible by the method for producing a plant plug, which method is described herein at another point.

As described, the plant plug comprises a growth medium according to the invention. Since the plant plug must have a solid shape for its handling and use, the growth medium present in the plant plug is solid, just like the plant plug itself. As already explained at another point, “solid” in the context of the invention means that the plant plug has a sufficient strength for handling by machine. At the same time, the plant plug is also not solid to the extent that plant root formation is prevented. Appropriate parameters for a suitable strength are already defined for the growth medium at another point and can equally apply to the plant plug.

The plant plug preferably consists of the growth medium. However, it is also conceivable that the plant plug consists of the growth medium only to an extent of 70% or more, preferably 80% or more, more preferably to an extent of 90% or more.

The size of the plant plug is matched with the size of the roots and the root network of a developing plant and with the intended use. In this connection, it is conceivable to form very small plant plugs which serve for cultivation up to a rooting phase/tissue culture phase in stage III for example. In addition, it is also possible to produce relatively large plant plugs, for example those which contain recesses for smaller plant plugs and can be used in a “plug-in-plug” system. The plant plug can therefore have, for example, a size of 0.125 cm³ or greater, preferably 1 cm³ or greater. In other words, the plant plug preferably has a volume of from 0.125 cm³ to 27 cm³, more preferably from 1 cm³ to 10 cm³. Since the plant plugs according to the invention are particularly suitable for automated high-throughput cultivation, the plant plugs can be particularly small in some embodiments. Therefore, in some embodiments, the plant plug has a volume of 27 cm³ or lower, 16 cm³ or lower, preferably 10 cm³ or lower, more preferably 8 cm³ or lower.

At the same time, the plant plug can have the shape of a cuboid, for example the shape of a cuboid having substantially equal sides. However, the plant plug according to the invention is not limited to this shape. For example, it is likewise conceivable to form plant plugs having a cylindrical or hemispheric shape.

As already explained above, the growth medium can comprise a water-insoluble component having a stabilizing action. The statements there relating to the growth medium are transferable to the plant plug. For instance, the plant plug can comprise a growth medium comprising a water-insoluble component, the outer volume of the plant plug being reduced by less than 60% in the case of a dissolution and removal of the gelling agents agar and carrageenan owing to a stabilizing action of the water-insoluble component.

In another aspect, the invention provides a method for producing a plant plug, preferably a plant plug for the automated cultivation of plants, characterized in that it comprises steps in which

(a) a growth medium according to the invention is provided in a liquid state;
(b) the growth medium is gelled in the shape of a plant plug; and
(c) optionally the growth medium is sterilized.
The method serves especially for the production of a plant plug according to the invention.

In a step (b) of the method according to the invention, the growth medium is gelled in the shape of a plant plug. To this end, the liquid medium provided in step (a) can be filled into an appropriate mold. The mold can, for example, be filled by pouring or pumping. The mold can be a mold having one or more depressions having the shape of a plant plug. Preferably, the mold is having multiple depressions, for example 16 or more, 48 or more. In this way, it is possible to produce plant blocks comprising multiple plant plugs. The plant plugs within the blocks are preferably initially connected to one another by gelled growth medium, but can be separated from one another, for example by cutting. Therefore, in a further aspect, the invention is directed to plant blocks, it being possible for the plant blocks to comprise 16 plant plugs or more, preferably 48 or more.

After the growth medium has been filled into the desired mold, the previously liquid growth medium is gelled, especially the gelling agents agar and carrageenan that are present in the growth medium. Suitable methods for gelling agar and carrageenan are known to experts and also described herein at another point. Preferably, the growth medium is sterilized and gelled simultaneously. For instance, the growth medium can be gelled (and simultaneously sterilized) in step (b) by steam pressure sterilization for example (at about 121° C. for example). In this embodiment, gelling step (b) and sterilization step (c) are carried out simultaneously. Alternatively, the gelling can, however, also be carried out at lower temperatures as explained herein at another point. In the gelling in step (b), the growth medium assumes the shape of a plant plug, since it is situated in the corresponding mold.

Whereas steps (b) and (c) can be carried out simultaneously as described above, it is also possible to carry out the steps successively. In this case, step (c) can, depending on the sterilization method selected, be carried out before or after the gelling in step (b). It is, for example, possible to carry out step (c) with the aid of a sterile-filtration before step (b) or to carry it out with the aid of a microwave sterilization, steam pressure sterilization or irradiation after step (b). If it is not necessary for the plant plug to be sterile, it is also possible to omit step (c).

In yet a further aspect, the invention provides a method for the automated cultivation of plants, characterized in that it comprises steps in which
(i) a plant plug according to the invention is contacted with a plant or a plant part;
(ii) plant plug and plant or plant part are cultivated together under growth conditions suitable for the plant such that the plant or the plant part roots in the plant plug and a plant unit is formed by plant or plant part and plant plug; and
(iii) the plant unit is transferred in an automated manner into
   (iii.1) a recess in a larger plant plug;
   (iii.2) a cell in a cultivation tray; and/or
   (iii.3) a soil substrate.

The method according to the invention is especially suitable for the automated handling of the plant plugs and of the plants connected to the plant plugs. This means that the plant is grippable and further processible in an automatic manner, i.e., by machine, by means of the plant plug. Further processing encompasses, for example, the relocation of the plug into another vessel or substrate. Further automated steps are likewise conceivable, as carried out in steps iii.1 to iii.3. The automated handling is independent of the rooting of the plants, i.e., independent of the rooting stage of the plants.

The contacting of plant or plant part and plant plug encompasses mere laying, pressing and/or insertion of plant or plant parts into the plant plug. Preferably, the stalk of the plant or of the plant part is gently pushed into the plant plug. In any case, the contact established in step (i) is sufficiently firm for the plant or the plant part to be able to start to form roots in the plant plug. In this connection, the term “plant part” refers especially to plant clones which were generated by division of a parental plant.

Thereafter, the plant or the plant part with the plant plug are cultivated together. This leads to growth processes, especially the formation of roots and possibly side shoots, and to the growth of leaves and upper shoot parts. Suitable growth conditions are known to experts and dependent on the nature of the plant to be cultivated. A plant unit arises, then, just from the first roots formed by the plant in the plant plug. In particular, the plant unit is characterized in that the cohesion between plant/plant part and plant plug is firmer than immediately after the application/contacting of the two.

The plant unit thus formed can be used further in different ways. It can be directly transferred into another cultivation element, for example into a recess in a larger plant plug or into a soil substrate, or transferred into a cell on a cultivation tray. This can serve for transport and/or for short-term storage.

Lastly, in a further aspect, the invention also provides for the use of a growth medium and/or plant plug according to the invention for the cultivation of plants, preferably for the automated cultivation of plants.

The invention also provides a plant unit consisting of a plant plug according to the invention and one or more plants rooted therein, the plants being in this case preferably present in tissue culture phase III (stage III).

### BRIEF DESCRIPTION OF THE FIGURES

Exemplary embodiments of the invention are depicted schematically in the drawings, where:

FIG. 1 shows various embodiments of the plant plug (12) according to the invention that together with a shoot (10) forms a plant unit (8), wherein the plant plug (12) has a cylindrical (A), cuboidal (B) or hemispheric (C) shape. In the embodiment shown in FIG. 1(A), a plant slit (16) was introduced into the plant plug (12), into which the shoot (10) was inserted. The shoots (10) in FIGS. 1(B) and (C) were gently pushed into the surface of the plant plug (12).

FIG. 2 shows the plant units (8) as per FIG. 1, wherein the shoot (10) has formed a root system (14) in the respective plant plugs (12).

FIG. 3 shows the automatic transfer of a rooted plant unit (8) according to the invention (cf. FIG. 3(A)), wherein the plant unit (8) was inserted by means of a gripper (20) into a recess into a larger plant plug that is not according to the invention, wherein the larger plant plug consists of a soil substrate (18) (cf. FIG. 3(B)). The gripper (20) comprises a gripper head (22) having two blades or lances, wherein, as a result of piercing of the blades or lances into the plant plug (12), a sufficiently good connection between gripper (20) and plant plug (12) is created for the plant unit (8) to be able to be transported by means of the plant plug.

FIG. 4 shows a plant block (24) according to the invention consisting of 48 plant units (8) as per FIG. 1(B).

FIG. 5 shows examples of plant plugs according to the invention without a shape-stabilizing component containing rooted Amelanchier (Chuckley pears);

FIG. 6 shows examples of plant plugs according to the invention without a shape-stabilizing component containing rooted Auricula (primula);

FIG. 7 shows examples of plant plugs according to the invention with the polyurethane foam BVB Sublime as shape-stabilizing component containing rooted Echinacea (coneflowers);

FIG. 8 shows examples of plant plugs according to the invention with the polyurethane foam Oasis as shape-stabilizing component containing rooted Leontopodium (edelweiss); and

FIG. 9 shows examples of plant plugs according to the invention with polyurethane foam and coconut fibers as shape-stabilizing components containing rooted Heuchera (coral bells).

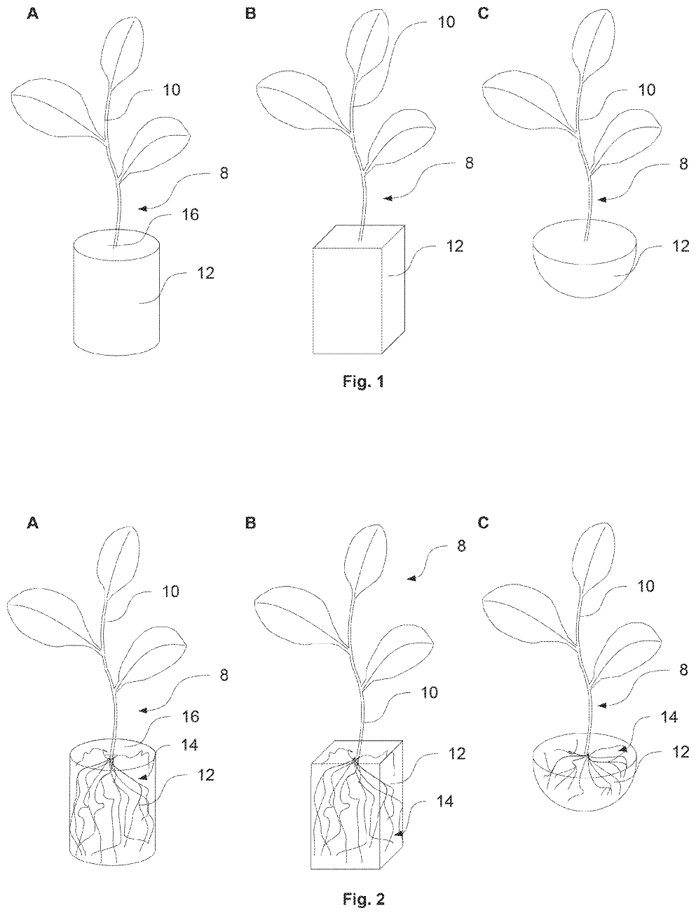

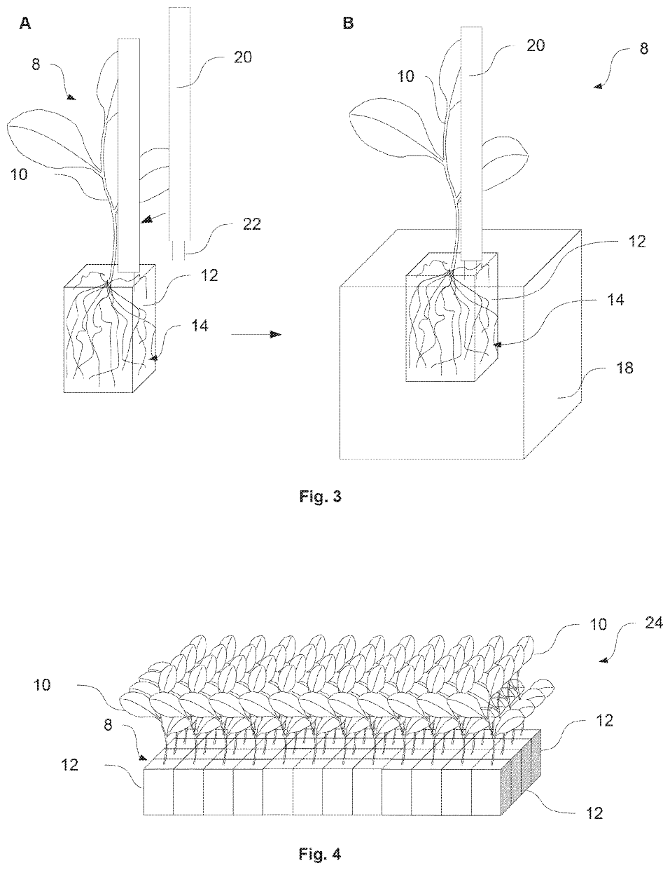

### DETAILED DESCRIPTION OF PREFERRED EMBODIMENTS

Further advantages, characteristics and features of the present invention become clear in the following detailed description of exemplary embodiments on the basis of the appended drawings. However, the invention is not limited to said exemplary embodiments.

### EXAMPLES

### Example 1: Production of a Plant Plug Using Luffa as Shape-Stabilizing Component

Technical Conditions for Producing a Plant Plug According to the Invention

The individual chemical components (macro-, micro-, vitamin components) were dissolved in distilled water and topped up to 75% of the amount to be boiled. Lastly, sugar and phytohormones were appropriately added. Appropriate concentration data for the different chemical components were gathered from the relevant literature (George, Edwin F., Puttock, David J. M., George, Heather J., 1987, Plant Culture Media, Volume I: Formulations and Uses, ISBN 0-9509325-2-3-Vol1, Exegetics Ltd., Edington, Westbury, Wilts. BA 13 4QG, England).

Then, the amount to be boiled was topped up to 100%. Thereafter, the pH was adjusted by dropwise addition of 1 N NaOH solution while stirring the growth medium. Finally, the appropriate concentration of gelling agents was added (6.4 g/l agar, CERO Agar Agar Powder, type 8925 X; 1.6 g/l carrageenan, CEROGEL Carrageenan (Danish agar agar), type 8886, 100% refined kappa-carrageenan). Either it was possible for the growth medium formulation to be filled into trays that were still nonsterile at this point for autoclaving (121° C., 15 min), or what was first carried out was steam pressure sterilization (121° C., 15 min), microwave sterilization, irradiation or sterile-filtration under constant mixing, and filling into sterile trays at a clean bench after the sterilization; to this end, the medium was cooled to 50-60° C.

As water-insoluble, stabilizing additive, dried luffa fruit was cut into pieces, the pieces having the size of a plant plug. The luffa pieces were then placed into the trays to be filled before said trays were filled with the growth medium.

After cooling, it was possible for the sterile vessels comprising trays and the plant plugs to be overlaid with plants for growth.

Overlaying of the Plant Plug with Plants

In this step, the plants were laid onto the plant plug for further growth or for rooting, either manually at a sterile work bench after division and possible shortening of leaves, cut into shape using scalpel and tweezers, or by a machine according to set-pattern programming using laser cutting or other automated division.

Culture Conditions Up Until Further Processing of the Plant in the Plant Plug

After overlaying, the plants grew in vessels (tray comprising individual cells based on plant plugs) under constant conditions in an artificially air-conditioned cultivation room. Said constant conditions included a uniform temperature (+/−1° C.), uniform lighting with defined light color, and uniform relative air humidity and regular air exchange.

Automatic Process for Transplanting the Plant Plug

The plant grew up to a (rooted) shoot in the plant plug in the culture room in accordance with the culture protocol. It was possible, then, for said plant to be automatically transplanted. Robotic processing was achieved by gripping the individual plant plug from above, at the side and/or by pushing up from below by means of a push-out mechanism. The grippers can be needles, sheets or other holders, and they can be made from different materials. The plant plug was then gripped together with the plant and put down into a new (larger) plant plug (e.g., nonsterile soil substrate) or an individual cell of a plant tray. This automatic transplantation process can proceed under either sterile or nonsterile conditions.

Further Processing of the Plant Plug in a Greenhouse

The described in vitro culture protocol ended with the adaptation of the plant in the plant plug to the nonsterile greenhouse conditions. In this step, the plant plug, consolidated with gelling agent and provided with a non-water-soluble component, together with the plant rooted into a soil-substrate plug.

### Example 2: Production of Further Plant Plugs According to the Invention

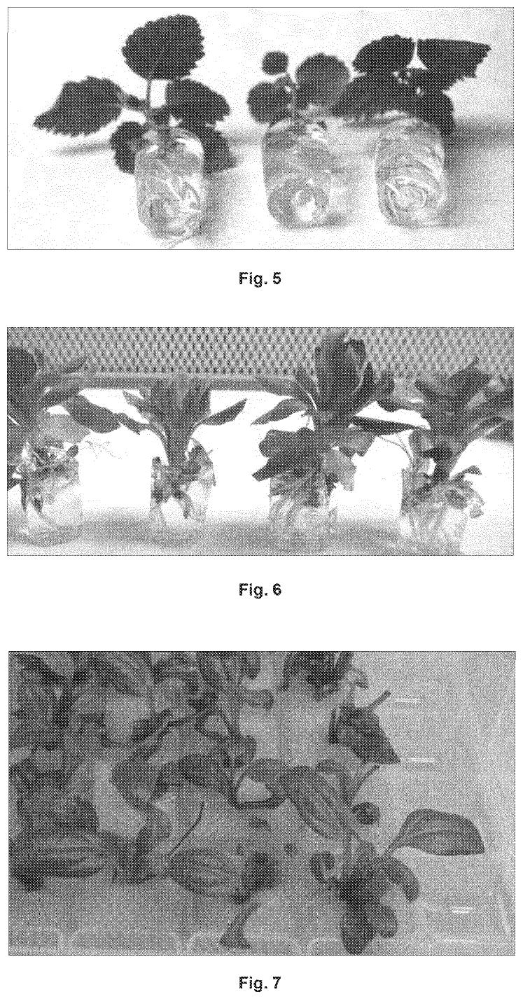

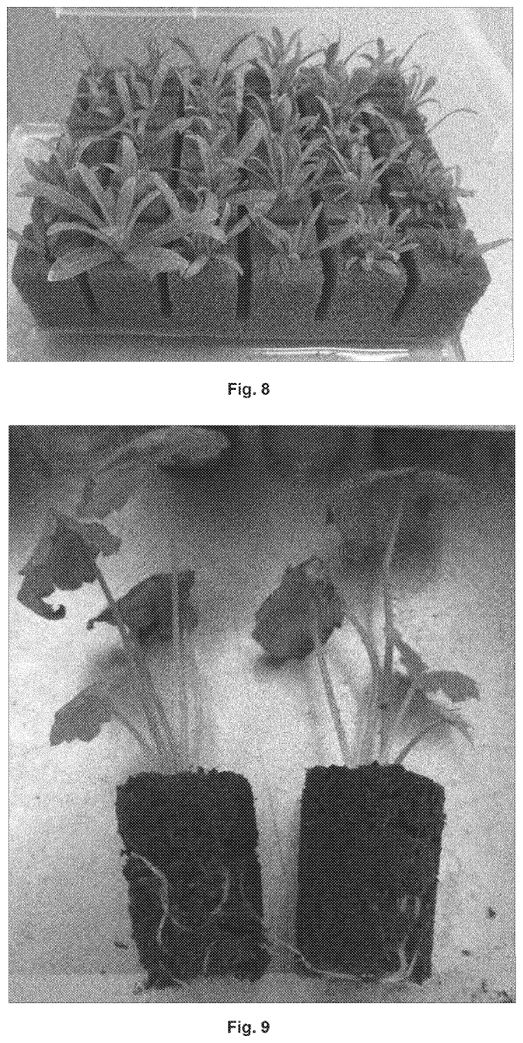

Analogously to the method described in Example 1, further plant plugs, in which luffa was not used as shape-stabilizing component, were produced and tested. The following plant plugs were produced and were tested with the following plants:

| Shape-stabilizing | | |
|---|---|---|
| component | Plant species | Result shown in |
| None | Amelanchier | FIG. 5 |
| None | Auricula | FIG. 6 |
| Polyurethane foam | Echinacea | FIG. 7 |
| (BVB Sublime) | | |
| Polyurethane foam | Leontopodium | FIG. 8 |
| (Oasis) | | |
| Polyurethane foam | Heuchera | FIG. 9 |
| and coconut fibers | | |

The polyurethane foams mentioned in the table were each used in concentrations of from 40% to 70% by weight, based on the plant plug; the coconut fibers were used in concentrations of from 5% to 15% by weight, based on the plant plug.

Good results (not shown) were also achieved using gelatin foam as shape-stabilizing component. What were added to an individual cell from in vitro trays were 4 mg/ml Gelatamp (Roeko) and 2 ml of liquid growth medium (cf. Example 1). The gelatin foam Gelatamp took up 0.8 ml of the liquid medium and the resultant plant plugs exhibited an adequate quality for automatic handling.

Although the present invention has been described in detail on the basis of exemplary embodiments, it is self-evident to a person skilled in the art that the invention is not limited to said exemplary embodiments, but that, on the contrary, modifications are possible such that individual features can be omitted or different combinations of the presented individual features can be realized, so long as there is no departure from the scope of protection of the appended claims. The present disclosure includes all combinations of the presented individual features.

### LIST OF REFERENCE SIGNS

8 Plant unit
10 Shoot
12 Plant plug
14 Root system
16 Plant slit
18 Soil substrate
20 Gripper
22 Gripper head
24 Plant block

1. A growth medium for the cultivation of plants, the growth medium comprising:
(a) 3 g/l to 18 g/l agar; and
(b) 0.5 g/l to 3 g/l carrageenan.

2. The growth medium as claimed in claim 1, further comprising:
(c) a shape-stabilizing component.

3. The growth medium as claimed in claim 2, wherein the shape-stabilizing component is a water-insoluble component.

4. The growth medium as claimed in claim 3, wherein the water-insoluble component is selected from the group consisting of plant parts, plastics, minerals, and mixtures thereof, and materials produced therefrom.

5. The growth medium as claimed in claim 4, wherein the plant parts are jute, hemp, flax, and/or coconut fibers.

6. The growth medium as claimed in claim 4, wherein the plastic is polyurethane foam.

7. The growth medium as claimed in claim 2, wherein the shape-stabilizing component is a water-soluble component.

8. The growth medium as claimed in claim 1, comprising agar in a concentration of from 5 g/l to 12 g/l.

9. The growth medium as claimed in claim 1, comprising carrageenan in a concentration of from 1 g/l to 2.2 g/l.

10. The growth medium as claimed in claim 1, wherein the growth medium has a gel strength of from 400 to 1200 g/cm².

11. A plant plug for the cultivation of plants, preferably for the automated or semiautomated cultivation of plants, wherein the plant plug comprises a growth medium comprising:
(a) 3 g/l to 18 g/l agar; and
(b) 0.5 g/l to 3 g/l carrageenan.

12. The plant plug as claimed in claim 11, wherein the plant plug has a volume of from 2 cm² to 20 cm².

13. The plant plug as claimed in claim 12, wherein the growth medium in the plant plug comprises a water-insoluble component and an outer volume of the plant plug is reduced by less than 60% in the case of a dissolution and removal of the agar and carrageenan owing to a stabilizing action of the water-insoluble component.

14. A method for producing a plant plug, preferably a plant plug for the automated or semiautomated cultivation of plants, comprising
(a) providing a growth medium in a liquid state; and
(b) gelling the growth medium in the shape of a plant plug.

15. A method for the automated or semiautomated cultivation of plants, comprising
(i) contacting a plant plug with a plant or a plant part;
(ii) cultivating the plant plug and plant or plant part together under growth conditions suitable for the plant such that the plant or the plant part roots in the plant plug and a plant unit is formed by plant or plant part and plant plug; and
(iii) the plant unit is transferred in an automated manner into
(iii.1) a recess in a larger plant plug;
(iii.2) a cell in a cultivation tray; and/or
(iii.3) a soil substrate.

16. The growth medium as claimed in claim 2, wherein the shape-stabilizing component is a water-soluble component selected from the group consisting of gelatin and hydroxyalkylcellulose.

17. The method for producing a plant plug as claimed in claim 14, wherein the growth medium comprises:
(a) 3 g/l to 18 g/l agar; and
(b) 0.5 g/l to 3 g/l carrageenan.

18. The method for producing a plant plug as claimed in claim 14, further comprising:
(c) sterilizing the growth medium.

19. The method for the automated or semiautomated cultivation of plants as claimed in claim 15, wherein the plant plug comprises a growth medium comprising:
(a) 3 g/l to 18 g/l agar; and
(b) 0.5 g/l to 3 g/l carrageenan.
